(19) United States
(12) Plant Patent Application Publication
(10) Pub. No.: US 2010/0306890 P1
(43) Pub. Date: Dec. 2, 2010

(54) Katsura Tree Named 'Biringer'

(50) ;

(75) Inventor: Josef Biringer, Mt. Vernon, WA (US)

Correspondence Address: Michelle Bos Legal LLC, P.O. Box 803, Zillah, WA, 98953, US

(73) Assignee: Biringer Nursery, LP, Mt. Vernon, WA (US)

(21) Appl. No.: 12/454,943

(22) Filed: May 26, 2009

(51) Int. Cl. A01H 5/00

(52) U.S. Cl. PLT/216

(57) ABSTRACT

‘Biringer’ Katsura is a newly and distinct variety of Katsura tree (Cercidiphyllum japonicum) having characteristics that are of commercial value in the nursery trade. As compared to Standard Katsura, ‘Biringer’ Katsura has darker green summer foliage; narrower, more upright branch angles; a tendency to hold the leaves later in the autumn; and greater winter hardiness.

### GENUS AND SPECIES

Cercidiphyllum japonicum

### VARIETY DENOMINATION

‘Biringer’

### BACKGROUND OF THE NEW VARIETY

The inventor discovered a single unusual plant in a row of Katsura trees at his commercial nursery in Mount Vernon, Wash. in July 2003. This plant exhibited narrower, more upright branch angles and darker green foliage than the surrounding Standard Katsura plants (not patented). This single plant was marked and observed during the remainder of 2003 and through 2004. In August 2004, 200 to 300 trees were propagated by chip budding and by cuttings (mist propagation). The propagated trees were planted in 2005. All trees from this propagation appeared identical to the original tree. Since 2005, approximately 500 trees have been propagated from the second-generation trees, and all are identical, confirming the trueness to type of the new variety. The new Katsura plant has been given the name ‘Biringer.’

### BRIEF SUMMARY OF THE INVENTION

The distinguishing characteristics of the ‘Biringer’ Katsura as compared to Standard Katsura are as follows:
1. Narrower, more upright growth habit.
2. Less vigor, by approximately 25-30%.
3. Darker green summer foliage.
4. Delayed senescence of leaves in the autumn, retaining foliage approximately two weeks longer.
5. Greater winter hardiness.

### DESCRIPTION OF THE DRAWINGS

FIG. 1 One-year shoots of Katsura, showing difference in senescence between ‘Biringer’ Katsura (top) and Standard Katsura (bottom).

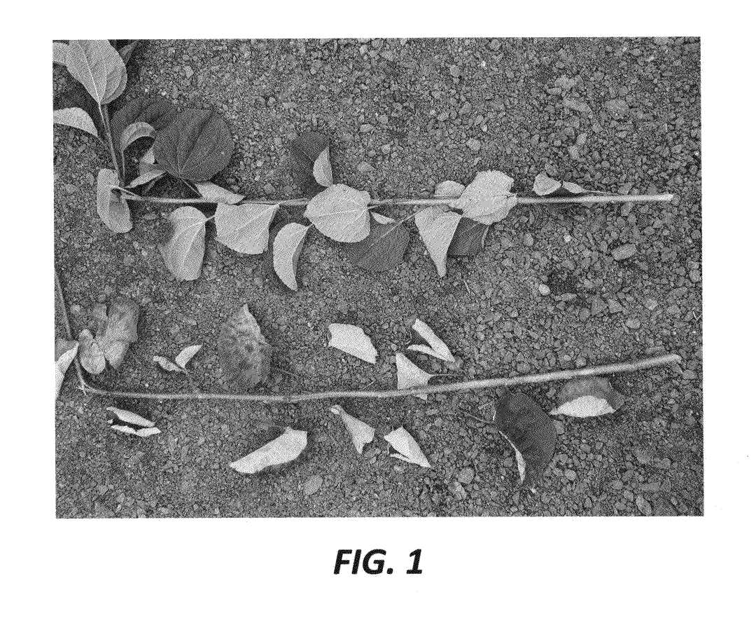

FIG. 2 Two-year old Katsura trees in nursery row. ‘Biringer’ Katsura is in the foreground on the left; Standard Katsura is on the right.

FIG. 3 Three year old ‘Biringer’ Katsura trees in nursery row.

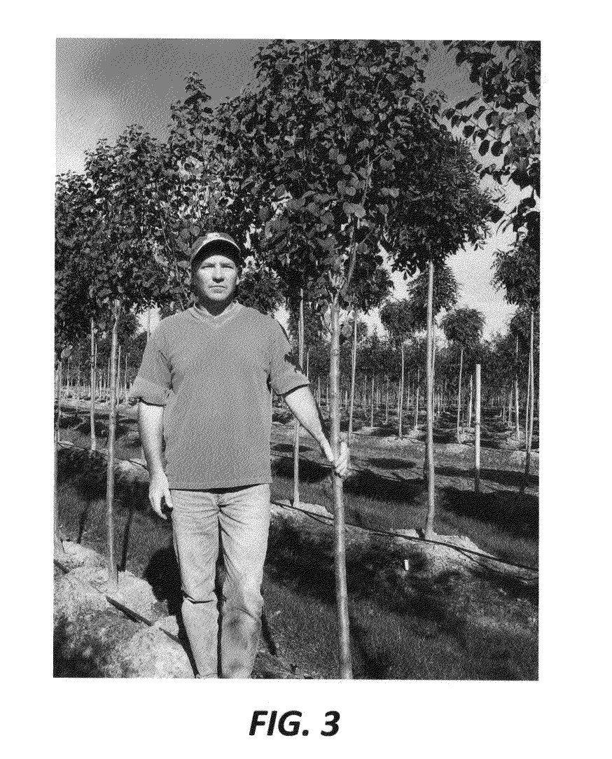

FIG. 4 Three year old Standard Katsura trees in nursery row.

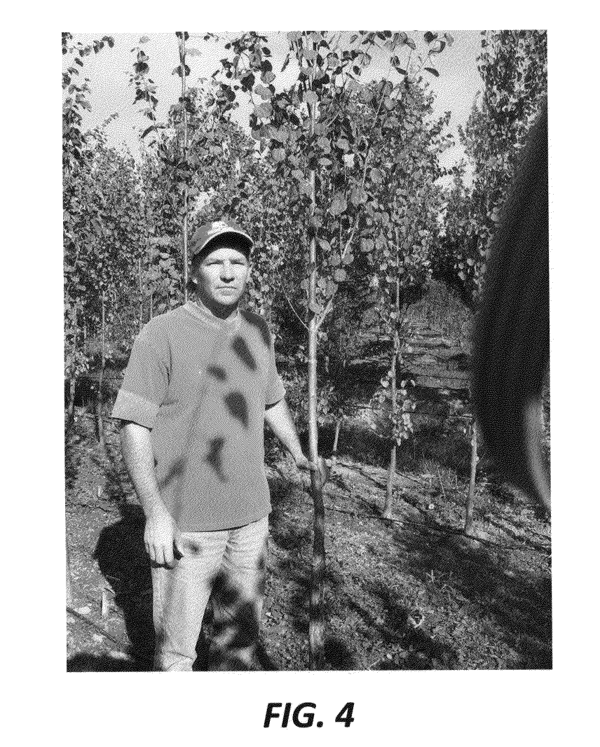

FIG. 5 Four year old ‘Biringer’ Katsura trees in nursery row.

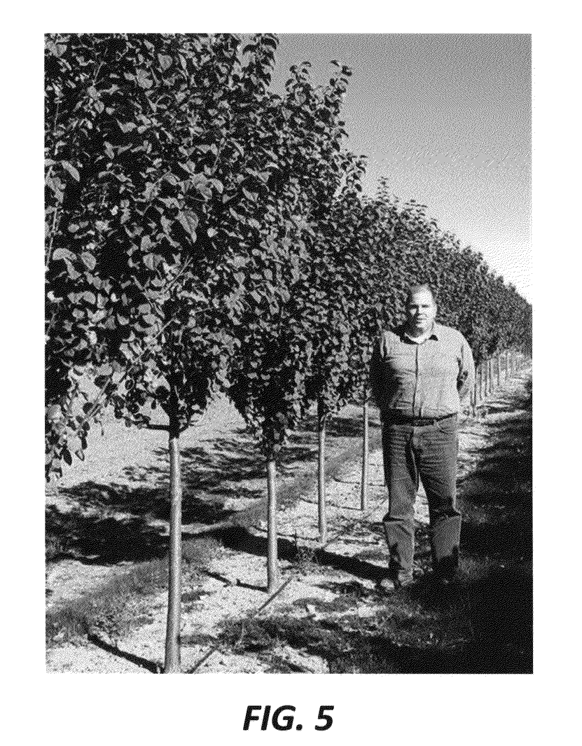

### DETAILED BOTANICAL DESCRIPTION

The standard Katsura tree, Cercidiphyllum japonicum, Siebold and Zucc. ex J. Hoffm. and H. Schult., is described in Hortus Third (MacMillan) as follows:
Upright-branching tree to 100 ft., trunk often branched above the base; leaves orbicular to ovate, to 4 in. long and cordate, crenate-serrate, glabrous, dark green above, glaucescent beneath, becoming yellow or scarlet in autumn; flowers before leaves; follicles about ¾ in. long.

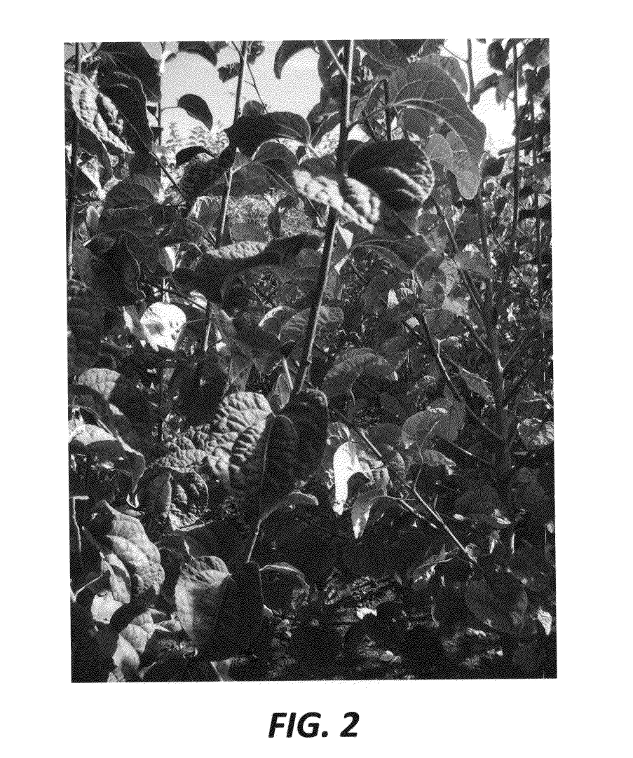

The detailed description which follows documents the differences between the Standard Katsura tree and the claimed ‘Biringer’ Katsura tree. This description was prepared from observation and sampling of two year old trees at Mount Vernon, Skagit County, Wash. in October 2008. Certain characteristics of this variety, such as growth and color, may change with changing environmental conditions, e.g., photoperiod, temperature, moisture, soil conditions, nutrient availability, or other factors. Color descriptions and other terminology are used in accordance with their ordinary dictionary descriptions unless the context clearly indicates otherwise. Color designations (hue/value/chroma) are made with reference to the Munsell Book of Color, Kollmorgen Instruments Corp., 405 Little Britain Road, New Windsor, N.Y. 12553.
Tree:
   Height.—Similar to that of Standard Katsura, approximately 5 feet at end of first season in the nursery (FIG. 2).
   Width.—First year 12 to 18 inches, approximately 6 inches narrower than Standard Katsura.
   Overall shape.—Upright branching tree. ‘Biringer’ has more upright growth habit and reduced vigor compared to Standard Katsura, thus appearing more compact.
Trunk:
   Texture.—Smooth.
   Color.—Medium brown, (7.5 YR 5/4). No difference in trunk bark texture or color from that of Standard Katsura.
Branches:
   Habit.—Upright branching, with side branches averaging 30 degrees from the vertical, compared to 45 degrees from the vertical in Standard Katsura.
   Size.—Somewhat more slender and with about 30% less vigor than Standard Katsura.
   Color.—Olive brown, (2.5 Y 4/4), as compared to light brown (10 R 4/4) in Standard Katsura.
   Lenticels.—Round to oblong, inconspicuous, light tan (7.5 YR 6/2), less than 1 mm in length and diameter.
   Internode length.—5.5 cm, as compared to 6.0 cm in Standard Katsura.
Leaves: Based on observation of 10 leaves on Oct. 11, 2008.
   Size.—6.5 cm long and 6.5 cm wide, similar to Standard Katsura.
   Form.—Orbicular to ovate, similar to Standard Katsura.
   Color.—Dark green (2.5 G 3/4) above, lighter green (2.5 G 6/8) beneath. Upper surface of leaves on Standard Katsura, when sampled, were more yellowish (2.5 GY 4/4), showing signs of senescence.
   Marginal form.—Crenate and glabrous above, glaucescent beneath, similar to that of Standard Katsura.
   Petiole.—2.7 cm in length, 2 mm in width, color reddish (2.5 R 3/10), as compared to a more purple color (2.5 R 3/4) in Standard Katsura.
Flowers
   Blossoms.—No flowers have been observed. The inventor has not observed flowers on Katsura trees. The literature suggests that Katsura trees typically produce separate male and female flowers in spring before foliation.
   Seeds.—None observed.
Other distinguishing characteristics:
Hardiness: On Nov. 20, 2006, a low temperature of 12 degrees Fahrenheit was reached in an area where Standard and ‘Biringer’ Katsura trees were growing. By the following spring it was observed that more than 50% of the Standard Katsura trees died, while all of the sixty plants of ‘Biringer’ cv. of the same age survived undamaged.
Disease/insect resistance and susceptibility: No observable difference in adaptability to various soil conditions, disease and insect infestation, or fall coloring have been observed between ‘Biringer’ Katsura and Standard Katsura.

I claim:

1. A new and distinct variety of Katsura tree substantially as described herein, having the following important characteristics as compared to Standard Katsura: more upright form, darker green summer foliage, narrower branch angles; a tendency to retain foliage later in the autumn, and greater winter hardiness.
